(19) United States
(12) Patent Application Publication
(10) Pub. No.: US 2006/0241443 A1
(43) Pub. Date: Oct. 26, 2006

(54) Real time ultrasound monitoring of the motion of internal structures during respiration for control of therapy delivery

(76) Inventors: Willet F. Whitmore III, Longboat Key, FL (US); Arthur J. Schenck, Fall City, WA (US); Gary M. Onik, Orlando, FL (US)

Correspondence Address: STEPTOE & JOHNSON LLP, 1330 CONNECTICUT AVENUE, N.W., WASHINGTON, DC, 20036, US

(21) Appl. No.: 11/281,416

(22) Filed: Nov. 18, 2005

Related U.S. Application Data
(60) Provisional application No. 60/629,403, filed on Nov. 22, 2004.

(51) Int. Cl. A61B 8/00; A61H 1/00

(52) U.S. Cl. 600/439; 601/2

(57) ABSTRACT

A method of targeting therapy such as radiation treatment to a patient includes: identifying a target lesion inside the patient using an image obtained from an imaging modality selected from the group consisting of computed axial tomography, magnetic resonance tomography, positron emission tomography, and ultrasound; identifying an anatomical feature inside the patient on a static ultrasound image; registering the image of the target lesion with the static ultrasound image; and tracking movement of the anatomical feature during respiration in real time using ultrasound so that therapy delivery to the target lesion is triggered based on (1) movement of the anatomical feature and (2) the registered images.

### CROSS-REFERENCE TO RELATED APPLICATIONS

The benefits of Provisional Application No. 60/629,403 filed Nov. 22, 2004 are claimed under 35 U.S.C. § 119(e), and the entire contents of this application are expressly incorporated herein by reference thereto.

### FIELD OF THE INVENTION

The invention relates to the application of continuous ultrasound imaging during the delivery of therapy to eliminate the errors induced by respiratory movement. More particularly, the invention relates to precision targeting of radiation therapy.

### BACKGROUND OF THE INVENTION

In the field of radiation oncology, tumors of all types are treated with ionizing radiation that disrupts the basic chemistry of tumor cells in order to cause cell death. The radiation may be delivered by either an invasive procedure that uses an implanted or temporary internal source, or non-invasively by an external source such as a linear accelerator. During treatment, a sufficient dose of ionizing radiation preferably is delivered to the tumor cells to prevent successful cellular replication or cause cell death while minimizing similar damage to surrounding normal cells and cell structures. For example, using implanted sources, this is typically achieved using multiple low energy pellets emitting radiation with a limited half life and tissue penetration. The pellets are carefully placed within the desired anatomical area to achieve a total tumor dose within the therapeutic range that matches the volume and shape of the tumor.

External beam irradiation (ERT), on the other hand, typically uses a high energy radiation source that penetrates entirely through the body. In order to treat an internal tumor without burning the overlying skin or normal structures, the beam typically is rotated around the patient using multiple angles of approach that can achieve a generally spherical or elliptical area of effective radiation dose within the body. Techniques also have been developed for shaping the treatment volume. These include varying the intensity of the beam by shading and filtering (intensity modulated radiation therapy, known as IMRT), and shaping the span and outline of the beam using similar methods. All these approaches currently take advantage of image guidance provided by one or more imaging modalities such as computed axial tomography (CT), magnetic resonance imaging (MR), positron emission tomography (PET) and ultrasound (U/S) to improve the accuracy of tumor targeting to huge beneficial effect. In addition, a great variety of medical products have been developed to enable accurate and reproducible patient positioning for ERT. For example, special treatment tables may be provided with reference marks for correlation with targeting images obtained from CT, combination CT and PET or MR scanners. These correlations provide a method for indexing of the patient and target lesion using these images to a treatment beam. The treatment tables may be provided with mounting points for custom, individually fitted, head, extremity and body conforming products for precise, repeatable positioning of the body part containing the targeted tumor area. Other prior art navigational and positioning systems for precise and reproducible targeting include markers fixed to the patient or implanted within the patient that may be detected in different ways. In combination, these advances in positioning, imaging/targeting and shaping of the treatment volume have dramatically improved treatment effectiveness while reducing the injury to adjacent tissues.

However, much of the accuracy attainable with these products is lost when treating any tumor or lesion in a body region that moves with respiration.

It is well known that the chest and abdominal organs can move several inches during respiration. Such movement can be especially problematic in a patient who is breathing voluntarily and not intubated with an endotracheal tube where volume input may be controlled. Normal respiration is a complex mixture of diaphragmatic and chest wall movements that may vary from breath to breath, and the result is that even a similar breath may not result in a similar position of internal organs that move with respiration. These variables have made accurate targeting of tumors that move with respiration a persistent problem. As a consequence, in typical prior art treatment methods, the loss of accuracy has been addressed simply by enlarging the treatment field to include the entire excursion area of the moving target throughout respiration. The additional morbidity and reduced treatment effectiveness using this method have been accepted in the past as being unavoidable.

More recently, techniques sometimes described as “respiratory gating” or “gating the beam” have been developed to trigger the treatment beam intermittently during a chosen phase of respiration when the target lesion is within a smaller known treatment area. This has been accomplished by various techniques for correlating target location with respiratory phase and volume and then monitoring respiration by using volume measurements, and by position sensors or reflectors of many types (e.g., infrared or visual sensors/reflectors) that are placed on the chest wall. To further improve accuracy, additional techniques including using implanted markers with intermittent x-ray (fluoroscopy) imaging and implanted markers that are tracked using electromagnetic methods have been developed. Because of the inconsistency of normal respiration, fundamental inaccuracies inherent in breath measurement, and the significant expense and/or the invasiveness required by these different “gating” methods, none of these approaches has achieved universal acceptance. The optimized therapy that is regularly achieved for static organs generally is not being achieved in the cases where respiratory movement affects the treatment field.

For example, U.S. Pat. No. 6,731,970 B2 to Schlossbauer et al. is directed to a method for breath compensation in radiation therapy. The patent discloses that the movement of the target volume is detected and tracked in real-time by deducing the current position of the target volume from a positional association between parameters that may simply be detected during the treatment, such as an implanted coil that may be tracked electromagnetically. Or, a roundabout route is taken, i.e. during radiation the target volume inside the patient is not itself tracked, but rather another parameter is measured which can be detected quite easily and which changes in constant relation to the changes in location of the target volume inside the patient. From this relatively stable relation, the respective location of the target volume inside the patient may be determined in real-time by establishing the position relationship of this second parameter with easily monitored respiratory parameters. A “reference breath” is chosen, and then the radiation is coordinated therewith. One method cited as potentially applicable for use in establishing this relationship is 3-D U/S. However, there is no suggestion to use U/S images to directly “gate” or time the treatment delivery. In general, methods that employ U/S specifically have not been used except for static positioning and targeting. The patent also discloses that the real time and reference breath actions are continuously compared to each other for the whole duration of the treatment. Where there is a difference between the breath phases, the patient is influenced such that the breath phases correspond again, and this may be done, for example, by supplying signals (preferably automatically) to the patient, to return him/her to the correct breath phase.

U.S. Patent Application Publication No. 2004/0081269 A1 to Pan et al. is directed to retrospective respiratory gating for imaging and treatment. In particular, the publication discloses a method for synchronizing images of a patient obtained via an imaging system using respiratory gating. The scanned images are synchronized with selected phases of a particular physiological characteristic, namely a respiratory cycle, to compensate for respiratory motion. Images are acquired while a patient is breathing normally and the breathing rhythm is recorded simultaneously with the image data. For synchronization purposes, a reference point is selected as one of either a minimum or a maximum in each respiratory cycle based on the application. An algorithm is used to assign phases to data points in the respiratory cycle with respect to a selected reference point. There is no suggestion, however, to use U/S to directly “gate” or time the treatment delivery.

U.S. Patent Application Publication No. 2002/0115923 A1 to Erbel is directed to a method for determining a current lung filling extent and method for assisting radiation therapy during respiratory shifting of the radiation target. The patent discloses a method for determining the filling of a lung, wherein the movement of an anatomical structure which moves during breathing, or one or more points on the moving anatomical structure whose movement trajectory is highly correlated with lung filling, is detected with respect to the location of at least one anatomical structure which is not spatially affected by breathing, and wherein each distance between the structures is assigned a particular lung filling value. Thus, a parameter is used which is not distorted by external references slipping, which describes the patient's breathing unambiguously, and which may be detected by measurement at reasonable expense. The distance provides such a parameter, from which it is possible to track breathing precisely, at any point in time, i.e. to positively determine lung filling at any time, even when detected at different times.

Also, Varian Medical Systems has marketed automated tools for real-time tumor tracking and respiratory gating during image-guided radiation therapy. In particular, Varian's On-Board Imager™ system was developed to synchronize image acquisition with a patient's respiratory cycle and automate “marker matching” for more precise tumor targeting. Gold seeds or other fiducial markers implanted in a tumor are used. The imager detects variations in marker position, and repositions the patient so that the tumor is directly in line with the radiation beam.

U.S. Patent Application Publication No. 2004/0116804 A1 to Mostafavi is directed to a method and system for radiation application. According to the published application, during gating simulation the movement of one or more landmarks or markers on the patient's body is optically measured using a camera. The detected motion of the landmark or marker results in the generation of motion signals. While motion data is being collected, a fluoroscopic video system generates imaging data for the tumor or tissue that is targeted for irradiation. The fluoroscopic image data and the marker motion data are recorded simultaneously on a common time base. The positional geometry of the fluoroscopic imaging system is configured to correspond to the projection geometry of the radiation beam source that will be used in applying radiation beams for treatment. This allows accurate simulation of the target volume to be achieved during actual treatment. During the planning phase of treatment, the fluoro video of the targeted body part or location, e.g., a tumor or in cases where the tumor is not visible in the fluoroscope image another anatomical landmark whose motion is highly correlated with the tumor, can be displayed in synchronization with the display of the motion signals. Simultaneous display of both sets of data allow a visual manner of determining the proper boundaries of the treatment intervals, based upon the range of movements of the targeted body part, location, tissue or tumor during particular portions of the motion signals. A visual display border can be formed around region(s) of interest in the fluoro video. The recorded fluoro image allows digital analysis and quantification of the amount of tumor motion resulting from regular physiological movement. The analysis can be performed by edge detection and tracking. This applies to anatomic landmarks, such as the diaphragm supporting the lungs, which show an intensity edge in the fluoroscope images. The edge position, and its rate of change, is used to select the optimum treatment interval. Also according to the published application, the treatment table upon which the patient is resting is configured such that it can be moved between the MRI device and the radiation therapy device. Moving the entire treatment table to move the patient between medical devices, rather than moving just the patient, reduces the chance that internal organs within the patient will shift during movement. There is no suggestion, however, to use U/S to directly “gate” or time the treatment delivery.

Despite these developments, there remains a need for a more accurate, more direct and non-invasive apparatus and method that addresses the challenges associated with accurately delivering radiation treatment to an anatomical region that is moving with respiration. In particular, there remains a need for a an apparatus and methods that addresses the challenges associated with accurately delivering ERT to an anatomical region that is moving with respiration.

### SUMMARY OF THE INVENTION

The invention relates to a device and method for using ultrasound imaging to perform “respiratory gating” or “gating of the beam” during the delivery of ERT. In particular, an ultrasound transducer is held in a fixed relationship to a patient, and in accordance with the method, the position of a target or target surrogate area is monitored in real time using ultrasound imaging during ERT targeting and treatment delivery. In one preferred embodiment, an ultrasound transducer is held by a bracket that is fixed to a manually positionable, lockable arm that, in turn, is attached to or otherwise maintainable in fixed relationship to a support surface such as a treatment table. When a patient is satisfactorily positioned on the support surface in a stable manner for treatment, the ultrasound transducer may be placed against the patient. Then, using the real time image, a 2-D scan plane of the target lesion or a surrogate anatomic target that moves with respiration in a similar and consistent manner as the target may be visualized during respiration. Within this image an easily tracked anatomic feature (AF) is chosen as the marker that will be used for timing of the treatment gate (e.g., signaling that the target is located within the desired treatment window and perfectly vis-a-vis the beam). The transducer positioning arm is locked to hold this view throughout the treatment period. Patient position and centering of the treatment beam may then proceed using CT or any other imaging, positioning or targeting modality.

In the case of CT, an ultrasound “snapshot” of the chosen AF for tracking is obtained at the same time interval as the targeting and centering CT images are obtained (while the patient is holding his breath). In this manner, the ultrasound images of the AF to be tracked correlate with the CT images that show the simultaneous anatomic position of the target area. Although it logically may be assumed that the respiratory phase and position (volume) will be the same each time the AF is in the same location in the real time ultrasound image as it was when the “snapshot” image was taken, the actual respiratory condition is not relevant to this method of gating. This method is completely reliant on the constant or consistent anatomic relationship (spacing) of the target region and the AF throughout respiration. Clearly, this relationship is most critical at the moment when the AF is in the operator selected, narrowly defined position very close to (if not exactly at) the position that is identified by the ultrasound “snapshot” image acquired precisely during the targeting CT images. Next, when the patient is moved out of the CT scanner and radiation treatment is started, the treatment beam may be triggered to accurately treat the desired target by continuously comparing the real time ultrasound image with the “snapshot” image of the AF, and firing the beam when they coincide. Preferably, this timing will assure that the treatment beam will be perfectly centered on the target lesion as shown on the CT image taken at the same relative anatomic position. For example, a repeatably verifiable anatomical feature such as the edge of an organ, a thick-walled artery, a small stone or a cyst associated with an organ, may be nearby and/or anatomically attached to the target lesion with respect to respiratory motion. Visualizing such an AF and comparing its appearance and location on the real time screen with the static “snapshot” image on the second half of a split screen may be used to trigger the treatment beam by medical personnel or by an astute and educated patient. More preferably, a detection algorithm may be focused on the AF by defining a region of interest and may be used to emit an electrical or optical signal when the AF is located in a defined position that closely coincides with its location shown or identified in the “snapshot” image. This signal may be programmed to transmit only on the inspiratory phase of respiration, only on the expiratory phase, or on both. A similar process using ultrasound as a real time tracking device may be applied using any other imaging modality for targeting. The simplest case is when the ultrasound device and images are also the targeting tool.

In some embodiments, a method employing sequential acquisition of two-dimensional (2D) ultrasound images is used to locate and establish the AF.

The invention relates to a method of targeting radiation therapy to a patient including: acquiring a first image(s) of a target lesion using computed axial tomography; acquiring a reference image using ultrasound simultaneously with acquisition of the first image(s), the reference image being one taken from a reference organ selected from the group consisting of a surrogate organ and the target organ; selecting a portion of the reference image that includes a repeatably identifiable anatomic feature of the reference organ; correlating the first image(s) with at least one selected from the group consisting of the reference image and a portion of the reference image; aiming radiation delivery using the first image(s); acquiring additional live images of the reference organ using ultrasound during delivery of radiation to the patient; delivering radiation to the target organ when a portion of the live or real time ultrasound image matches the selected portion of the reference ultrasound image.

The invention also relates to computer executable software code stored on a computer readable medium, the code comprising code to acquire or signal the time of acquisition of a first image(s) of a target lesion using computed axial tomography; code to acquire a reference image using ultrasound simultaneously with acquisition of the first image(s), the reference image being of a reference organ selected from the group consisting of a surrogate organ and the target organ; code to select a portion of the reference image that includes a repeatably identifiable anatomic feature of the reference organ; code to correlate the first image(s) with at least one selected from the group consisting of the reference image and a portion of the reference image; code to aim radiation delivery using the first image(s); code to acquire additional live images of the reference organ using ultrasound during delivery of radiation to the patient; code to deliver radiation to the target organ when a portion of the live image matches the selected portion of the reference ultrasound image.

In addition, the invention relates to a programmed computer system for targeting radiation therapy to a patient comprising at least one memory having at least one region storing computer executable program code and at least one processor for executing the program code stored in said memory, wherein the program code includes code to acquire or signal the time of acquisition of a first image(s) of a target lesion using computed axial tomography; code to acquire a reference image using ultrasound simultaneously with acquisition of the first image(s), the reference image being of a reference organ selected from the group consisting of a surrogate organ and the target organ; code to select a portion of the reference image that includes a repeatably identifiable anatomic feature of the reference organ; code to correlate the first image(s) with at least one selected from the group consisting of the reference image and a portion of the reference image; code to aim radiation delivery using the first image(s); code to acquire additional live images of the reference organ using ultrasound during delivery of radiation to the patient; code to trigger the delivery of radiation to the target organ when a portion of the live image matches the selected portion of the reference ultrasound image. In one preferred embodiment this code would be integral within an ultrasound machine.

Moreover, the invention relates to a method of timing the exposure of radiation treatment to a patient including: identifying a target lesion inside the patient on a computed axial tomography image(s); identifying an anatomical feature inside the patient on a static ultrasound image acquired simultaneously; registering the computed axial tomography image with the static ultrasound image; tracking movement of the anatomical feature during respiration in real time using ultrasound so that radiation delivery to the target lesion is triggered based on (1) movement of the anatomical feature and (2) the registered images.

The invention further relates to computer executable software code stored on a computer readable medium, the code comprising code to signal or time the acquisition of targeting images of a target lesion inside the patient on a computed axial tomography, magnetic resonance or other targeting image(s); code to identify an anatomical feature inside the patient on a real time ultrasound image; code to register the time of acquisition of the targeting image(s) with a simultaneously acquired static ultrasound image; code to track movement of a selected anatomical feature during respiration in real time using ultrasound so that radiation delivery to the target lesion is triggered based on (1) movement of the anatomical feature and (2) the registered images.

The invention also relates to a programmed computer system for targeting radiation treatment of a patient comprising at least one memory having at least one region storing computer executable program code and at least one processor for executing the program code stored in said memory, wherein the program code includes code to identify the time of acquisition of an image of a target lesion inside the patient on a computed axial tomography image or other image targeting modality; code to identify a selected anatomical feature inside the patient on a static ultrasound image obtained simultaneously with the targeting image; code to register the computed axial tomography image with the static ultrasound image acquired in the same time interval; code to track movement of the anatomical feature during respiration in real time using ultrasound so that radiation delivery to the target lesion is triggered based on (1) movement of the anatomical feature and (2) the registered images. In one preferred embodiment this code would be integral within an ultrasound machine.

Also, the invention relates to a method of targeting therapy such as radiation treatment to a patient including: identifying a target lesion inside the patient using an image obtained from an imaging modality selected from the group consisting of computed axial tomography, magnetic resonance tomography, positron emission tomography, and ultrasound; identifying an anatomical feature inside the patient on a static ultrasound image; registering the image of the target lesion with the static ultrasound image; tracking movement of the anatomical feature during respiration in real time using ultrasound so that radiation delivery to the target lesion is triggered based on (1) movement of the anatomical feature and (2) the registered images.

The invention further relates to computer executable software code stored on a computer readable medium, the code including: code to identify a target lesion inside a patient using an image obtained from an imaging modality selected from the group consisting of computed axial tomography, magnetic resonance tomography, positron emission tomography, and ultrasound; code to identify an anatomical feature inside the patient on a static ultrasound image; code to register the image of the target lesion with the static ultrasound image; code to track movement of the anatomical feature during respiration in real time using ultrasound so that radiation delivery to the target lesion is triggered based on (1) movement of the anatomical feature and (2) the registered images.

And, the invention relates to a programmed computer system for targeting therapy such as radiation treatment to a patient including at least one memory having at least one region storing computer executable program code and at least one processor for executing the program code stored in said memory, wherein the program code includes code to identify a target lesion inside a patient using an image obtained from an imaging modality selected from the group consisting of computed axial tomography, magnetic resonance tomography, positron emission tomography, and ultrasound; code to identify an anatomical feature inside the patient on a static ultrasound image; code to register the image of the target lesion with the static ultrasound image; code to track movement of the anatomical feature during respiration in real time using ultrasound so that radiation delivery to the target lesion is triggered based on (1) movement of the anatomical feature and (2) the registered images.

Although the embodiments described herein are described as using computed axial tomography imaging, it should be understood that the embodiments described herein may be applied to any imaging system and radiation treatment system suitable to the desired purpose.

### BRIEF DESCRIPTION OF THE DRAWING

Preferred features of the present invention are disclosed in the accompanying drawing, wherein:

FIG. 1 shows a block schematic diagram of a representative system according to one preferred exemplary embodiment of the present invention with a CT imaging system, an ultrasound imaging system, and a radiation treatment system.

### DETAILED DESCRIPTION OF THE PREFERRED EMBODIMENTS

As used herein, the term “organ” is used in its broadest sense to mean any tissue or organ including a normal or pathological cell type such as a cancer cell, in which case the selected organ or tissue can be a primary tumor or a metastatic lesion. Non-limiting examples of organs and tissues include kidney, heart, liver, lung, spleen, small and large bowel, gallbladder, pancreas, adrenal glands, lymph nodes, ovary, bone marrow, and neuronal tissue.

As used herein, the term “hollow organ” means an organ of a subject's body which depends for its principal function upon its ability to receive and/or act as a conduit for fluid contents. A hollow organ typically is in fluid communication with another hollow organ and/or with the outside of the body. Many organs of the gastrointestinal and genitourinary tracts are classified as hollow viscus organs. These include stomach, gall bladder, uterus, and bladder. Other hollow organs which act more as fluid passageways include esophagus, small and large intestines, hepatic ducts, cystic duct, common bile duct, pancreatic duct, heart, veins, arteries, vagina, uterine (i.e., Fallopian) tubes, ureters, and urethra. In the case of a stomach being the hollow organ, “fluid contents” includes any of the following: masticated food, imbibed liquid, chyme, gastric mucus, gastric acid, and other gastric secretions. In other contexts “fluid contents” can also include other body fluids such as intestinal contents, bile, exocrine pancreatic secretions, blood, and urine. Moreover, in the case of a lung being a hollow organ, “fluid contents” means gas such as air.

As used herein, the term “target lesion” is defined as an anatomical structure or region to receive treatment such as radiation treatment.

As used herein, the term “target organ” refers to an organ with a target lesion.

As used herein, the term “anatomical feature” (AF) is defined as an anatomical structure or region that moves in a proportional, equivalent, or predictable manner with respect to a target lesion during respiration. The anatomical feature may be proximate the target lesion, such as when the anatomical feature is a feature of the liver and the target lesion is a lesion of the liver. The anatomical feature may be remote from the target lesion such as when the anatomical feature is a feature of the liver and the target lesion is a lesion of the lung. Examples of anatomical features include the edge of an organ, a thick-walled artery, a small stone or a cyst associated with an organ.

As used herein, the term “surrogate” used in the context of an anatomical feature refers to an anatomical structure or region remote from a target lesion, but physically associated in terms of movement so that it moves proportionally and consistently with the target lesion throughout respiration.

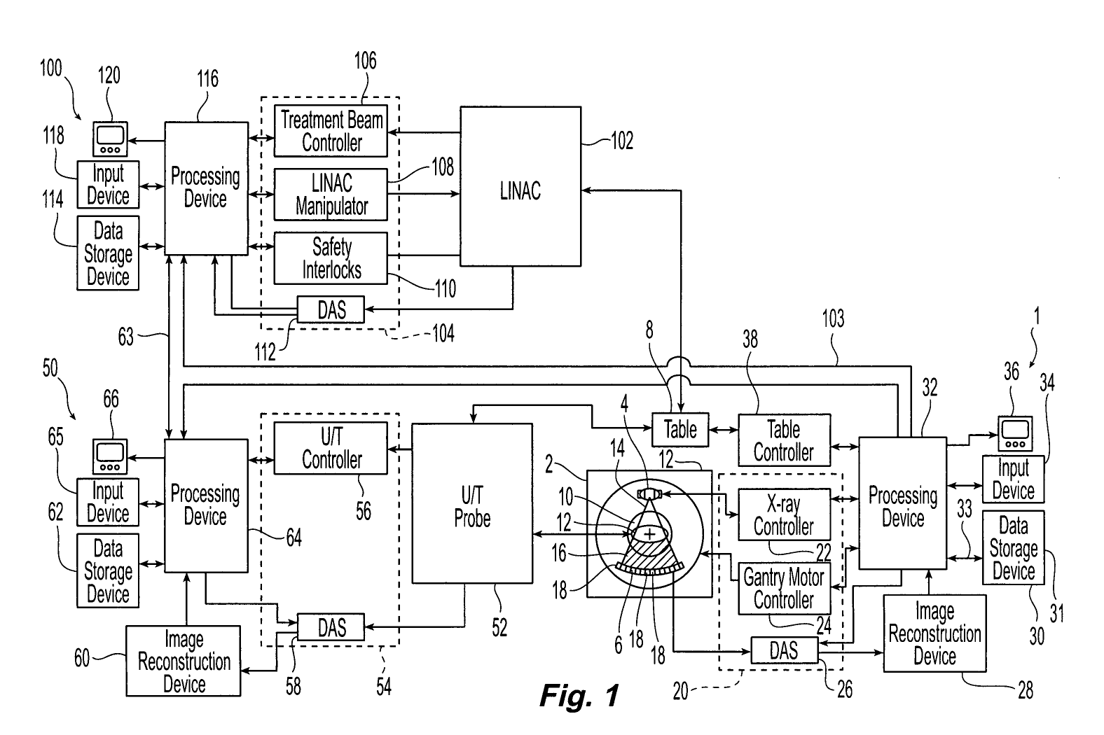

Referring to FIG. 1, a representative system according to one preferred exemplary embodiment of the present invention has a CT imaging system 1, an ultrasound imaging system 50, and a radiation treatment system 100.

A representative CT imaging system 1 as known in the art is disclosed in U.S. Patent Application Publication No. 2004/0081269, the entire contents of which are expressly incorporated herein by reference thereto. In summary, a CT imaging system 1 may include, but is not limited to, a gantry 2 having an x-ray source 4, a radiation detector array 6, a patient support structure 8 and a patient cavity 10, wherein x-ray source 4 and radiation detector array 6 are opposingly disposed so as to be separated by patient cavity 10. A patient 12 may be disposed upon patient support structure 8, which is then disposed within patient cavity 10. X-ray source 4 projects a x-ray radiation beam 14 toward radiation detector array 6 so as to pass through patient 12. Radiation beam 14 may be collimated so as to lie within an X-Y-Z volume of a Cartesian coordinate system referred to as an “imaging volume.” After passing through and becoming attenuated by patient 12, attenuated x-ray beam 16 is received by radiation detector array 6. Radiation detector array 6 includes, but is not limited to a plurality of detector elements 18 wherein each of the detector elements 18 receives attenuated x-ray beam 16 and produces an electrical signal responsive to the intensity of attenuated x-ray beam 16. X-ray source 4 and radiation detector array 6 may be communicated with a control mechanism 20 associated with CT imaging system 1.

Control mechanism 20 controls the rotation and operation of x-ray source 4 and/or radiation detector array 6. Control mechanism 20 includes, but is not limited to, an x-ray controller 22 communicated with x-ray source 4, a gantry motor controller 24, and a data acquisition system (DAS) 26 communicated with radiation detector array 6, wherein x-ray controller 22 provides power and timing signals to x-ray source 4, gantry motor controller 24 controls the rotational speed and angular position of x-ray source 4 and radiation detector array 6 and DAS 26 receives the electrical signal data produced by detector elements 18 and converts this data into digital signals for subsequent processing. CT imaging system 1 may also include an image reconstruction device 28, a data storage device 30 and a processing device 32, wherein processing device 32 is communicated with image reconstruction device 28, gantry motor controller 24, x-ray controller 22, data storage device 30, an input device 34 and an output device 36. Moreover, CT imaging system 1 may also includes a table controller 38 communicated with processing device 32 and patient support structure 8, so as to control the position of patient support structure 8 relative to patient cavity 10.

A patient 12 may be disposed on patient support structure 8, which is then positioned by an operator via processing device 32 to be disposed within patient cavity 10. Gantry motor controller 24 is operated via processing device 32 to cause x-ray source 4 and radiation detector array 6 to rotate relative to patient 12. X-ray controller 22 is operated via processing device 32 so as to cause x-ray source 4 to emit and project a collimated x-ray beam 14 radiation beam 14 toward radiation detector array 6 and hence toward patient 12. X-ray radiation beam 14 passes through patient 12 so as to create an attenuated x-ray beam 16, which is received by radiation detector array 6.

Detector elements 18 receive attenuated x-ray beam 16, produces electrical signal data responsive to the intensity of attenuated x-ray beam 16 and communicates this electrical signal data to data acquisition system (DAS) 26. DAS 26 then converts this electrical signal data to digital signals and communicates both the digital signals and the electrical signal data to image reconstruction device 28, which performs high-speed image reconstruction. This information is then communicated to processing device 32, which stores the image in data storage device 30 and displays the digital signal as an image via output device 36.

As shown in FIG. 1, ultrasound imaging system 50 for example includes an ultrasound probe or transducer 52 for acquiring ultrasound data. In the preferred exemplary embodiment, transducer 52 is rigidly associated with support 8 such that transducer 52 may be positioned against a patient in a single location during simultaneous collection of CT data and ultrasound data, and then in the same location during simultaneous collection ultrasound data and delivery of radiation treatment. Control mechanism 54 controls transducer 52. Control mechanism 54 may include, but is not limited to, an ultrasound controller 56 and a DAS 58 communicated with transducer 52. DAS 58 receives the electrical signal data produced by transducer 52 and converts this data into digital signals for subsequent processing. Ultrasound imaging system 50 may also include an image reconstruction device 60, a data storage device 62 and a processing device 64, wherein processing device 64 is communicated with image reconstruction device 60, data storage device 62, an input device 65 and an output device 66.

In addition, as shown in FIG. 1, radiation treatment system 100 for example includes a beaming apparatus 102 such as a linear accelerator (LINAC) for generating a treatment beam. When apparatus 102 is activated, a collimated ionizing beam is emitted and directed at a target region of a patient for radiation treatment. Control mechanism 104 includes a treatment beam controller 106, a LINAC manipulator 108 such as a robot arm that positions the treatment beam with respect to the patient, and safety interlocks 110 to ensure that the beaming apparatus is not activated accidentally. A DAS 112 communicates with beaming apparatus 102 and receives the electrical signal data produced by apparatus 102 and converts this data into digital signals for subsequent processing. A data storage device 114 and a processing device 116 may be provided, wherein processing device 116 is communicated with data storage device 114, an input device 118 and an output device 120.

The preferred exemplary embodiment of the present invention may be embodied in the form of computer or controller implemented processes and apparatuses for practicing those processes. The present invention may also be embodied in the form of computer program code containing instructions embodied in tangible media, such as floppy diskettes, CD-ROMs, hard drives, or any other computer-readable storage medium 31, 62, 114, wherein when the computer program code is loaded into and executed by a computer or controller, the computer becomes an apparatus for practicing the invention. The present invention may also be embodied in the form of computer program code or signal such as signal 33, 63, 103 for example, whether stored in a storage medium 31, 62, 114 loaded into and/or executed by a computer or controller, e.g. processing device 32, 64, 116 or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

In the preferred exemplary embodiment, processing devices 64, 116 are integrated so that data from U/T probe 52 may be used to control the timing of radiation delivery from beaming apparatus 102. Moreover, processing device 32 may be integrated with processing device 116 and for example may be integrated for purposes of correctly timing ultrasound image acquisition to processing device 64. In particular, as described in detail below, beaming apparatus 102 is directed at a target region of a patient for radiation treatment, with the target region being identified on an image from CT imaging system 1. Image collection by CT imaging system 1 is synchronized with image collection by ultrasound imaging system 50.

Next, the combined use of CT imaging system 1, ultrasound imaging system 50, and radiation treatment system 100 according to a preferred exemplary embodiment of the present invention will be described. Patient imaging such as CT imaging may be undertaken remote from radiation treatment equipment. The patient typically is supported on a tabletop, tray, platform or other surface during imaging, and then moved to the radiation treatment area for example on a gurney. In accordance with the present invention, preferably the patient is supported on the same surface 8 (e.g., a movable tray) during imaging and radiation treatment, even though these procedures may occur in separate locations.

In accordance with a preferred exemplary method according to the present invention, a patient first may be fixed in position with respect to a support surface. Next, ultrasound may be used to scan the patient to identify a good reference image scan plane that shows the target lesion or a surrogate organ throughout respiration. Preferably, the reference image should have at least one clearly identifiable AF in the range of about 2 mm to about 10 mm in size.

Some organs may easily be observed by ultrasound imaging, while other organs or portions thereof may be difficult to adequately observe by ultrasound imaging. While the abdomen and pelvic region can be examined using ultrasound, other anatomical regions cannot effectively be imaged with this technique. Ultrasound, for example, may be used to examine the abdomen, liver, spleen, pancreas, gallbladder, kidneys, ovaries and uterus, and aorta and other abdominal arteries. However, ultrasound is not suitable for organs such as the lungs, stomach and intestines, which are partially filled with air and considered “opaque” to sound. Hard tissues such as bone suffer a similar imaging problem. Organs that cannot effectively be imaged with ultrasound, and concomitantly the organs that lie beneath them in the ultrasound imaging direction, thus cannot readily serve as organs to provide an AF for the method of the present invention, and in this case a surrogate must be used.

In the preferred exemplary embodiment, an ultrasound transducer 52 is held against the patient in a fixed position throughout the targeting procedure and radiation treatment procedure. This may be accomplished by coupling the transducer to a curvilinear articulating arm which in turn is coupled to the support surface that moves with the patient from the CT imaging to radiation treatment venues. Preferably, the transducer is attached to the free end of the arm, and movement of the arm is releasably lockable so that the position of the transducer against the skin of the patient may be fixed and remain the same during the CT imaging and radiation treatment. Alternatively, the transducer may be otherwise rigidly coupled to the patient as with a frame that is independent of the support surface on which the patient is disposed, the frame being coupled directly to the patient.

Next, with the patient positioned in the CT scanner, an image volume for purposes of treatment planning and targeting may be acquired at any chosen phase of inspiration or expiration. The ultrasound transducer remains in place against the patient during CT scanning, but preferably the transducer is not in the CT imaging plane. First, the patient is prepared for the simultaneous CT scanning and ultrasound data collection. The patient is instructed to hold his or her breath, typically for up to 5 seconds. While the patient is holding his or her breath, the ultrasound image acquisition occurs simultaneously with the image volume acquisition sequence for the CT scanner. The ultrasound image acquisition may be slaved to the CT acquisition trigger using a remote control, thus ensuring exact temporal alignment of the CT and ultrasound images. Typically however, this may be unnecessary because of the very short time interval required to obtain the ultrasound image that may be acquired at any time during the much longer interval required to obtain the CT images. Thus, the ultrasound image acquisition at this stage may be manually triggered while the longer CT imaging procedure occurs. Through the ultrasound image acquisition, a fixed ultrasound image or frame is recorded (the “snapshot” image), typically a 2-D image, preferably showing a repeatably verifiable anatomic feature (AF) of an organ such as the edge thereof, a thick-walled artery, or a stone or cyst associated with an organ. In the preferred exemplary embodiment, an ultrasound image may be taken containing an AF from a surrogate organ, but in alternate embodiments the ultrasound image may be taken of an AF within the target organ. CT imaging preferably specifically is taken of the target lesion for purposes of directing the treatment beam.

In one embodiment, a split-screen image display may be used. The ultrasound reference “snapshot” image may be viewed on one portion of the image display, while the real time ultrasound image that is used to track the AF may be viewed on another portion of the image display. The ultrasound image/data are preferably stored within the ultrasound machine and the CT image/slice/data are stored in the CT machine and interfaces with the ERT targeting machinery and software. The ultrasound and CT machines and their associated computers, processors, hardware, software, and systems may be integrated so that data and capabilities associated with one machine may be available to the other. However, this integration or connection is not essential, because the only information required from the CT machine is the time of acquisition of the targeting images. This is required because the ultrasound “snapshot” image must be acquired simultaneously.

Next, the patient is moved out of the CT scanner, while the ultrasound transducer remains in continuous, fixed and locked position relative to the patient. Ultrasound scanning may be temporarily discontinued.

While viewing the ultrasound reference image, a region of interest (ROI) containing part or all of the AF is defined on the image. Preferably, the ROI may be selected as a rectangular or circular region or other discrete portion of the ultrasound image, for example using ultrasound system software measurement tools. Preferably, the ROI circumscribes or otherwise surrounds the AF. The ROI is defined by the function T(x,y).

Ultrasound scanning is again commenced. Preferably while the patient takes normal breaths, real time imaging of the same anatomic view as the reference image will appear and a target detection process is initiated. In particular, a target identification scan is performed during real time imaging within the same (ROI) identified in the reference image. The ROIs of the reference image and the live image have the same spatial orientation since the ultrasound transducer has remained fixed in position. A target detection algorithm then may be used to match the AF within the ROI of the reference image to the AF within the ROI on the live images. In particular, when the AF transits the ROI on the live image, closely matching the image of the AF in the ROI on the reference image, the position of the target organ is known within very tight limits. Although the ROI itself may be used as the AF, more preferably, a target detection algorithm is applied to match a selected AF within the ROI of the reference image to the same AF, in the same position within the ROI, when displayed on the live images. Preferably, the algorithm may be used to provide a signal as the AF transits the ROI and when the images are closely matched. A preferred exemplary target detection algorithm is as follows:

$$E = {\sum }_{x = 1}^{M} {\sum }_{y = 1}^{N} T ( x , y ) - F ( x , y ) \qquad Eq . ( 1 )$$

where:
   E is the composite error in gray scale between template (T) and image (F);
   M and N define the number of pixels in x and y, respectively; and
   T and F are the digitized gray scale values at each x and y location.

The computation described in equation (1) is performed by software by scanning through a pixel map of the (ROI) on successive image frames. The algorithm takes into account the shape, texture, and location of the target object within the ROI. This approach is more straightforward than conventional cross-correlation techniques used in digital image processing because such applications are designed to identify known targets at random locations within an image plane. Advantageously, because of the fixed placement of the patient and ultrasound transducer during CT imaging and ultrasound imaging, it is only necessary to look for the target at a specific location within the fixed ROI. The ROI has significantly less pixels to scan than the full image, and the algorithm is much faster and simpler computationally than conventional cross-correlation techniques.

At an image frame rate of 30 frames per second for example, the temporal resolution of image alignment is about 33 ms. Such high resolution may minimize errors caused by target velocity, and may allow sufficient time for computation even with relatively large ROI pixel maps. At each pixel location within the ROI, the gray scale difference is calculated. The differences are accumulated for each of the pixels within the ROI so the image frame may be identified where E is minimized.

In an ideal case E=0. However, because of very slight misalignments in patient position or organ motion throughout the respiratory cycle, it is unlikely that a perfect match would occur. To overcome this, and to make sure that the best match is detected within one image frame to avoid hysteresis effects, in the preferred embodiment it is recommended that the patient take one or two deep breaths to permit the system to self-calibrate by determining the minimum value of E. Then on the next respiratory cycle, the best match can be detected and automatic triggering of the therapy device can be initiated.

Although a preferred exemplary target detection algorithm is disclosed in Equation 1, other suitable algorithms may be used to achieve the desired targeting.

When the ultrasound system processor determines that a match has occurred between the position of a feature identified in the ROI of the reference image and the same feature's position on a live image, a signal may be sent to an output port that is electrically connected to a therapy device such as a radiation beam generator. The therapy device may be provided with a remote triggering capability slaved to the output of the ultrasound system. Triggering of the therapy device may occur within milliseconds of target identification, thereby ensuring that the therapy is delivered to the exact spatial location required.

Even if the AF were to move along a first plane while the target lesion moved in a second plane, the movement would still be predictable and correlated with the present invention.

Thus, through real time ultrasound imaging, a radiation treatment for example may be undertaken in which the effects of respiratory movement are minimized. The treatment thus may be targeted to the anatomical area of interest while avoiding the deleterious effects of radiation exposure to anatomical regions outside the zone of interest for the target organ.

### EXAMPLE 1

In an exemplary use of the method of the present invention, a lung lesion is to be treated with radiation therapy, particularly ERT. As described previously, the lung moves during the respiratory cycle, and thus it is desirable to irradiate the patient only to the extent that the radiation is directed at the lesion itself. The lung cannot be readily imaged using ultrasound, and thus this hollow organ cannot be imaged with ultrasound. In this case, the liver may be chosen as the surrogate organ and a suitable surrogate AF within an easily visualized area of the liver may be chosen, while the lung contains the target lesion. Because lung lesion movement may be correlated to movement of a surrogate AF within the liver as described above, the radiation beam may be triggered based on movement of the surrogate AF with respect to a reference image. Because the target lesion is in a known anatomic position when the surrogate AF is in its “snapshot” position, the beam may be timed and directed only to the target lesion of the target organ. This is done with no direct regard to respiration per se.

### EXAMPLE 2

In another exemplary use of the method of the present invention, a lesion of the liver is to be treated with radiation therapy, particularly ERT. As described previously, the liver moves during the respiratory cycle, and thus it is desirable to irradiate the patient only to the extent that the radiation is directed at the lesion itself. The liver may be readily imaged using ultrasound, simultaneously with radiation treatment of the liver, so long as the ultrasound transducer does not interfere with the field of the radiation treatment. No surrogate organ is needed. Because liver lesion movement may be tracked, the radiation beam may be triggered based on movement of the liver with respect to an AF within the liver chosen from a reference 2-D ultrasound image.

A representative computer system is now described in conjunction with which the embodiments of the present invention may be implemented. The computer system may be a personal computer, workstation, or a larger system such as a minicomputer. However, one skilled in the art of computer systems will understand that the present invention is not limited to a particular class or model of computer. A representative computer system includes a central processing unit (CPU), random access memory (RAM), read only memory (ROM), one or more storage devices, an input device, an output device, and a communication interface. A system bus is provided for communications between these elements. The computer system may additionally function through use of an operating system such as Windows, DOS, or UNIX, however one skilled in the art of computer systems will understand that the present invention is not limited to a particular configuration or operating system. Storage devices may illustratively include one or more floppy or hard disk drives, CD-ROMs, DVDs, or tapes. Input devices comprise a keyboard, mouse, microphone, or other similar device. Output devices comprise a computer monitor or any other known computer output device. The communication interface may be a modem, a network interface, or other connection to external electronic devices, such as a serial or parallel port. It should be noted that, although one computer terminal may be used, the system may be configured such that a plurality of computers are in communication with one another, and configured for parallel processing by such plurality of computers. Ideally this computer system will be closely integrated with the ultrasound image processing computer or preferably embedded within it.

The CPU is preferably linked to the RAM and ROM, either by means of a shared data bus, or dedicated connections. The CPU may be embodied as a single commercially available processor. Alternatively, in another embodiment, the CPU may be embodied as a number of such processors operating in parallel. The ROM is operable to store one or more instructions, discussed above in the context of the target detection algorithm, which the CPU is operable to retrieve, interpret and execute. The ROM preferably stores processes for searching and accessing a pixel map, as discussed above. In addition, the ROM may store processes for example to provide self-teaching and logic placement functions, provide a display from memory protocol, manage pixel map database updates, manage datacells, configure performance and stability attributes, and manage synchronization and recovery protocols. A CPU local memory storage device may be operable to provide high-speed storage used for storing temporary results and control information.

While various descriptions of the present invention are described above, it should be understood that the various features can be used singly or in any combination thereof. Therefore, this invention is not to be limited to only the specifically preferred embodiments depicted herein.

Further, it should be understood that variations and modifications within the spirit and scope of the invention may occur to those skilled in the art to which the invention pertains. For example, preferably in the present invention contra-recognition is employed with sequential acquisition of two-dimensional (2D) ultrasound images to locate and establish the AF. In an alternate embodiment, three-dimensional image processing may be used. Moreover, preferably in the present invention the ultrasound transducer is positionally fixed to the patient, but in alternate embodiments, the ultrasound transducer may be positionally registered in the treatment room for example to the CT scanner or radiation delivery equipment. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. A method of targeting therapy such as radiation treatment to a patient comprising:
identifying a target lesion inside the patient using an image obtained from an imaging modality selected from the group consisting of computed axial tomography, magnetic resonance tomography, positron emission tomography, and ultrasound;
identifying an anatomical feature inside the patient on a static ultrasound image;
registering the image of the target lesion with the static ultrasound image;
tracking movement of the anatomical feature during respiration in real time using ultrasound so that therapy delivery to the target lesion is triggered based on (1) movement of the anatomical feature and (2) the registered images.

2. The method of claim 1, wherein the movement of the anatomical feature is tracked using a target detection algorithm that correlates positions of the anatomical feature shown (1) on the static ultrasound image and (2) on live ultrasound images acquired during respiration in real time.

3. The method of claim 2, wherein the target detection algorithm comprises:
$$E = {\sum }_{x = 1}^{M} {\sum }_{y = 1}^{N} T ( x , y ) - F ( x , y )$$
where:
E is the composite error in gray scale between template (T) and image (F);
M and N define the number of pixels in x and y, respectively; and
T and F are the digitized gray scale values at each x and y location.

4. The method of claim 2, wherein the target detection algorithm accounts for shape, texture, and location of the anatomical feature.

5. The method of claim 1, wherein the anatomical feature has a dimension between about 2 mm and about 10 mm.

6. The method of claim 1, further comprising:
delivering radiation therapy to the target lesion.

7. The method of claim 1, wherein the static ultrasound image and images acquired in real time using ultrasound are all acquired using an ultrasound transducer disposed in substantially the same fixed position with respect to the patient.

8. The method of claim 7, wherein the ultrasound transducer is further disposed in substantially the same fixed position with respect to the patient when the target lesion is identified.

9. The method of claim 1, further comprising:
supporting the patient on a first support during acquisition of the ultrasound images and on a second support during delivery of radiation, the first and second supports being the same.

10. A method of targeting radiation therapy to a patient comprising:
acquiring a first image of a target lesion using computed axial tomography;
acquiring a reference image using ultrasound simultaneously with acquisition of the first image, the reference image being of a reference organ selected from the group consisting of a surrogate organ and the target organ;
selecting a portion of the reference image that includes a repeatably identifiable anatomic feature of the reference organ;
correlating the first image with at least one selected from the group consisting of the reference image and a portion of the reference image;
aiming radiation delivery using the first image;
acquiring additional live images of the reference organ using ultrasound during delivery of radiation to the patient;
delivering radiation to the target organ when a portion of the live image matches the selected portion of the reference image.

11. The method of claim 10, wherein a target detection algorithm is used to determine when the portion of the live image matches the selected portion of the reference image, the target detection algorithm comprising:
$$E = {\sum }_{x = 1}^{M} {\sum }_{y = 1}^{N} T ( x , y ) - F ( x , y )$$
where:
E is the composite error in gray scale between template (T) and image (F);
M and N define the number of pixels in x and y, respectively; and
T and F are the digitized gray scale values at each x and y location.

12. The method of claim 10, wherein the anatomic feature has a dimension between about 2 mm and about 10 mm.

13. The method of claim 10, wherein the ultrasound images are acquired using an ultrasound transducer retained in substantially fixed position with respect to the patient during the acquisition of the reference image and during the delivery of radiation to the patient.

14. The method of claim 13, wherein the ultrasound images are acquired using an ultrasound transducer retained in substantially fixed position with respect to the patient, and wherein the ultrasound transducer remains in substantially fixed position with respect to the patient during acquisition of the first image using computed axial tomography.

15. The method of claim 13, wherein the ultrasound transducer is disposed outside of an imaging plane of the computed axial tomography during acquisition of the first image.

16. The method of claim 10, wherein the first image and the reference image are acquired while the patient holds a breath.

17. A programmed computer system for targeting therapy such as radiation treatment to a patient comprising at least one memory having at least one region storing computer executable program code and at least one processor for executing the program code stored in said memory, wherein the program code comprises:
code to identify a target lesion inside a patient using an image obtained from an imaging modality selected from the group consisting of computed axial tomography, magnetic resonance tomography, positron emission tomography, and ultrasound;
code to identify an anatomical feature inside the patient on a static ultrasound image;
code to register the image of the target lesion with the static ultrasound image; and
code to track movement of the anatomical feature during respiration in real time using ultrasound so that therapy delivery to the target lesion is triggered based on (1) movement of the anatomical feature and (2) the registered images.

18. The system of claim 17, further comprising an imaging system comprising an x-ray source, a radiation detector array, and an x-ray controller.

19. The system of claim 17, further comprising an ultrasound imaging system comprising an ultrasound transducer and an ultrasound controller.

20. The system of claim 17, further comprising a radiation treatment system comprising a beaming apparatus and a treatment beam controller.
